(19) United States
(12) Patent Application Publication
(10) Pub. No.: US 2007/0282350 A1
(43) Pub. Date: Dec. 6, 2007

(54) Device for arranging wire leads from medical testing apparatus

(76) Inventor: Lynne J. Hernest, Hewitt, NJ (US)

Correspondence Address: CASELLA & HESPOS, 274 MADISON AVENUE, NEW YORK, NY, 10016, US

(21) Appl. No.: 11/809,526

(22) Filed: Jun. 1, 2007

Related U.S. Application Data
(60) Provisional application No. 60/811,043, filed on Jun. 5, 2006.

(51) Int. Cl. A61B 19/00

(52) U.S. Cl. 606/129; 600/382; 600/393

(57) ABSTRACT

A portable lead alignment device is provided for aligning, organizing and storing in a pre-assembled fashion the leads extending from an EKG apparatus. The device includes an elongate flexible support with at least one linear array of chest lead alignment tunnels disposed thereon. Each tunnel snugly accommodates a chest lead so that the lead can be slid longitudinally relative to the support. However, the tunnels prevent the leads from being separated from the device. The lead alignment device may further include a plurality of ground lead organizers for releasably retaining the ground leads in a neat looped arrangement on the support.

This application claims priority on U.S. Provisional Patent Appl. No. 60/811,043, filed Jun. 5, 2006.

### BACKGROUND OF THE INVENTION

1. Field of the Invention

The invention relates to a lightweight portable device for organizing and storing the conductive wire leads that extend from an EKG apparatus or similar medical apparatus.

2. Description of the Related Art

An electrocardiograph (EKG) machine is an apparatus that measures and records electrical currents associated with the activities of heart muscles. A medical doctor can analyze a graph produced by the EKG apparatus and can identify certain heart abnormalities. The EKG apparatus requires electrodes to be placed at selected locations on the patient. A typical EKG apparatus requires two electrodes (V1 and V2) to be placed symmetrically on the chest of the patient. Four more electrodes (V3-V6) then extend in spaced relationship around the left side of the patient. Thus, a total of six electrodes (V1-V6) extend generally transversely across and partly around the patient. Four ground electrodes then are connected respectively to the arms (LA, RA) and legs (LL, RL) of the patient. Each electrode is connected to a corresponding designated lead that extends from the EKG apparatus.

EKG tests provide critical medical information regarding heart function. These tests have wide application throughout the medical and insurance industries in both stationary settings and in mobile situations. Hospitals perform EKG exams as part of routine preadmission testing, throughout the hospital on in-patients when ordered by doctors, and in the emergency room when diagnosing suspected heart cases. Medical offices, especially cardiologists, frequently perform EKG tests. EMT (ambulance) squads often perform EKG exams when responding to emergency calls. EKG tests also frequently are part of a prerequisite physical exam for insurance coverage. These exams are performed on a mobile basis, usually at the client's home or workplace. Speed and accuracy are important factors in EKG tests. Portability also is an important issue for mobile EKG exams.

The EKG exam typically is performed by a medical technician who may be employed by the hospital, the doctor's office or working as an independent contractor for insurance physicals. Preparing the patient for the EKG test often is a very time consuming part of the exam. In particular, the medical technician must place the electrodes on the patient at the appropriate locations. The wire leads then must be extended carefully from the EKG apparatus to the appropriate electrodes. Frequently, the long wire leads from the EKG apparatus are tangled or in disarray after being disconnected from the previous patient since there is no mechanism for maintaining organization of the wires between uses. Thus, the medical technician must take time to carefully untangle and align the wires in proper order for connection to the appropriate electrodes. Improper connection of wires to the electrodes can result in erroneous EKG data that can be the basis for an improper diagnosis, medical treatment or insurance decision. The considerable time required for untangling, aligning and connecting the various EKG leads has a significant effect on the efficiency and productivity for the medical technician.

The prior art includes devices for aligning the wires extending from an EKG device. For example, U.S. Pat. No. 6,620,105 shows a wire alignment device specifically adapted for use with an EKG apparatus. Rigid plastic wire holders of the type shown in U.S. Pat. No. 6,620,105 and in the prior art wiring harness environments are not well suited for use in close proximity to a patient.

In view of the above, it is an object of the subject invention to provide a device for conveniently aligning the leads of an EKG apparatus.

Another object of the invention is to provide a device, which once assembled with the wire leads in a proper order, is ready for repeated use without disassembly and reassembly for each use.

An additional object of the invention is to provide easy reaching for lead positioning despite variations in the sizes and shapes of patients on whom the leads will be mounted

It is another object of the invention to provide a lead alignment device that can be used easily and comfortably in close proximity to a patient.

A further object of the invention is to provide an EKG device that can be collapsed and/or folded for easy storage and transportation, while still maintaining the preassembled order of the leads.

Still another object of the invention is to provide a lead alignment device for use with an EKG apparatus that is light weight and inexpensive.

Yet another object of the invention is to provide a device that can be used with different types of EKG devices having wire leads with different cross sections and/or differently configured or dimensioned connectors on the ends of the leads.

Another object of the invention is to provide a device that can be disassembled from a wire lead for maintenance or repair if necessary.

### SUMMARY OF THE INVENTION

The invention relates to a device for efficiently aligning the leads of an EKG apparatus. The device includes an elongate flexible support that may be formed from a synthetic fabric, natural fabric, extruded sheet material, leather or other soft, durable material that does not interfere with the electrical activity of the EKG test. The support preferably is long and narrow. The length of the support preferably is dimensioned to drape completely across the abdomen or pelvis of a typical patient in a medial-to-lateral (left-to-right) direction. The dimension of the support parallel to the height direction of the patient preferably is much less than the dimension of the support side-to-side or medial-to-lateral direction of the patient. However, the small dimension of the support preferably is selected to engage a sufficient longitudinal portion of each lead to ensure approximately parallel alignment of the leads.

The device further includes tunnel-like lead alignment means for snugly, but slidably, holding at least the chest leads associated with the EKG device so that the leads are substantially parallel to one another and spaced from one another. The snug retention of the leads prevents random unintended movement of the leads (e.g. in response to gravitational forces) while permitting controlled slidable movement of the leads along their axial directions in response to pulling forces by the technician. The lead alignment means may comprise at least one flexible strip that is releasably attachable to the flexible support. Preferably, the lead alignment means comprises first and second flexible strips disposed substantially in registration with one another so that the first flexible strip is secured to the support and the second flexible strip is releasably secured to the first flexible strip. Alternatives to the flexible strips may include: fabric loops, non-metallic eyelets, non-metallic clips or the like. First and second substantially linear arrays of lead alignment means preferably extend parallel to one another substantially along the medial-to-lateral direction of the patient and hence along the long dimension of the support. The lead alignment means in the first linear array substantially align respectively with lead alignment means in the second array. Each lead alignment means preferably is configured to permit the respective lead to move longitudinally along the lengths of the lead, and hence transverse to the medial-to-lateral direction of the support on the patient. However, each lead alignment means further is configured to prevent transverse movement of the leads along the medial-to-lateral direction of the support and to prevent unintended separation of the leads from the lead alignment device. However, each lead alignment means preferably is configured to permit the leads to be separated intentionally from the device to accommodate periodic replacement of the lead alignment device or periodic replacement or repair of a wire lead.

The lead alignment means used for the chest leads of the EKG apparatus generally do not contribute to efficient use of the ground leads of the EKG apparatus. Nevertheless, the ground leads can become entangled with one another or with the chest leads. Accordingly, the device of the subject invention may include ground lead retainers or organizers for releasably holding the ground leads in neat accessible coils. The ground lead retainers may include Velcro strips and may be positioned at the left and/or right sides of the support of the device.

The EKG leads are assembled into the device of the subject invention prior to use of the EKG device and will remain assembled in the device from one use to another. More particularly, the V1-V6 chest leads are inserted sequentially into dedicated V1-V6 lead alignment means that are arranged sequentially from the right side to the left side of the support (left-to-right order as the technician is looking at the patient). Each lead alignment means on the support is dimensioned to permit the lead to be slid longitudinally in the support without allowing the connector at the end of the lead to slide into or through the lead alignment means and without permitting unintended separation of the leads from the device. Additionally, each lead alignment means is configured to accommodate EKG leads of different cross-sectional dimensions that may be used by different manufacturers of EKG machines. Still further, the lead alignment means prevents movement of the connector through the lead alignment means despite the fact that the connector shape and size may vary from one manufacturer to another. Lead alignment means formed from mating pairs of Velcro strips work very well in that the Velcro strips can be secured to produce a tunnel of appropriate cross-section. Furthermore, the fabric of the Velcro strips exerts a controlled frictional resistance on the leads that avoids unintended movement of the leads in the tunnels produced by the Velcro strips. The ground leads may be looped or coiled after the V1-V6 leads have been secured. The conveniently looped or coiled ground leads then are held on the support by the above-described retainers or organizers. As a result, all leads are now assembled into their dedicated locations.

Prior to use, the chest leads (V1-V6) preferably are positioned relative to the device so that the connectors at the ends of the chest leads are substantially adjacent the device. The ground leads may be coiled loosely and secured to the ground lead retainers or organizers at the left and/or right sides of the support of the device. The device and the leads can be coiled or folded for convenient storage and transportation.

The medical technician uses the EKG device by unwrapping the wire leads and the lead alignment device. A gauze, pad, fabric or paper strip then can be placed on the abdomen of the patient below the location at which the EKG electrodes are affixed. The medical technician then unfolds the support and places the support on the gauze, pad or other sanitary item that optionally is placed across the abdomen of the patient. More particularly, the long dimension of the support is orientated in the medial-to-lateral or side-to-side direction of the patient so that the leads are on the side of the support facing away from the patient (towards the technician) and so that the connectors at the ends of the leads are facing towards the head of the patient. All electrodes are placed in the appropriate positions on the chest, arms and legs of the patient. If desired, this placement of the electrodes can be carried out before accessing the EKG device. The medical technician then sequentially grasps the connectors of the six chest leads and slides the respective chest leads through the lead alignment means so that the connectors at the ends of the leads can be moved towards and connected with the respective electrodes on the chest of the patient. The device prevents the leads from being entangled with one another and allows the connectors at the ends of the leads to align substantially with the electrodes to which the connectors are to be connected. The technician then releases the arm and leg ground leads from the retainers or organizers on the support and connects the connectors at the ends of the ground leads to the appropriate electrodes on the arms and legs of the patient. The connectors are mated with the respective electrodes in the known manner and the EKG test is performed in the known manner. At the end of the test, the medical technician merely disconnects the ground leads from the electrodes on the arms and legs of the patient and coils the ground leads. The coiled ground leads then are secured to the retainers or organizers on the support of the device. The technician then disconnects the six chest leads from their electrodes and pulls the chest leads back towards the device so that the connectors thereof substantially abut the device. Of course, the order of these steps can be varied in accordance with the preference of the technician, so that the chest leads may be disconnected first. The device and the leads then are rolled and/or folded for convenient disposition for storage or transportation.

The array of leads for an EKG test is not completely symmetrical. In particular, four of the leads extend around the left side of the patient. As a result, there is a potential that the non-symmetrical disposition of the leads can cause the device of the subject invention to shift on the patient. Accordingly, the right side of the support can be weighted slightly to substantially balance the non-symmetrical disposition of the leads. As a result, the device is less likely to slide laterally on the patient.

Most EKG tests are performed while the patient is in a supine position. However, some EKG tests can be performed while the patient is sitting. In these situations, the device can be placed on the lap of the patient. Alternatively, the device can be used with a shawl-like carrier or strap. The carrier may be U-shaped and can be draped around the neck, over the shoulders and towards the abdomen. The above-described flexible support can be releasably attached to the carrier by Velcro or other non-metallic attachments.

### BRIEF DESCRIPTION OF THE DRAWINGS

FIG. 1 is a perspective view of a device for arranging wire leads from a medical testing apparatus shown in proximity to the medical testing apparatus and stored in a carrying case.

FIG. 2 is a perspective view of the device for arranging wire leads independent of the medical testing apparatus and the carrying case.

FIG. 3 is a top plan view of the lead alignment device of the subject invention in a fully extended condition.

FIG. 4 is a cross-sectional view taken along line 44 in FIG. 3.

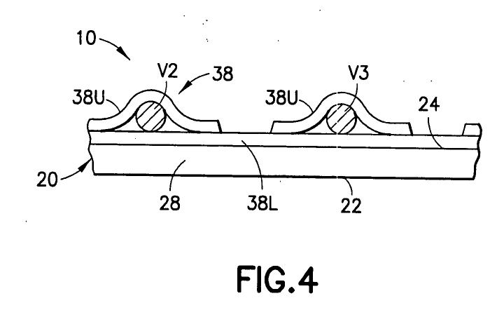

FIG. 5 is a perspective view of the device for arranging wire leads positioned on a patient but prior to attaching the leads to the patient.

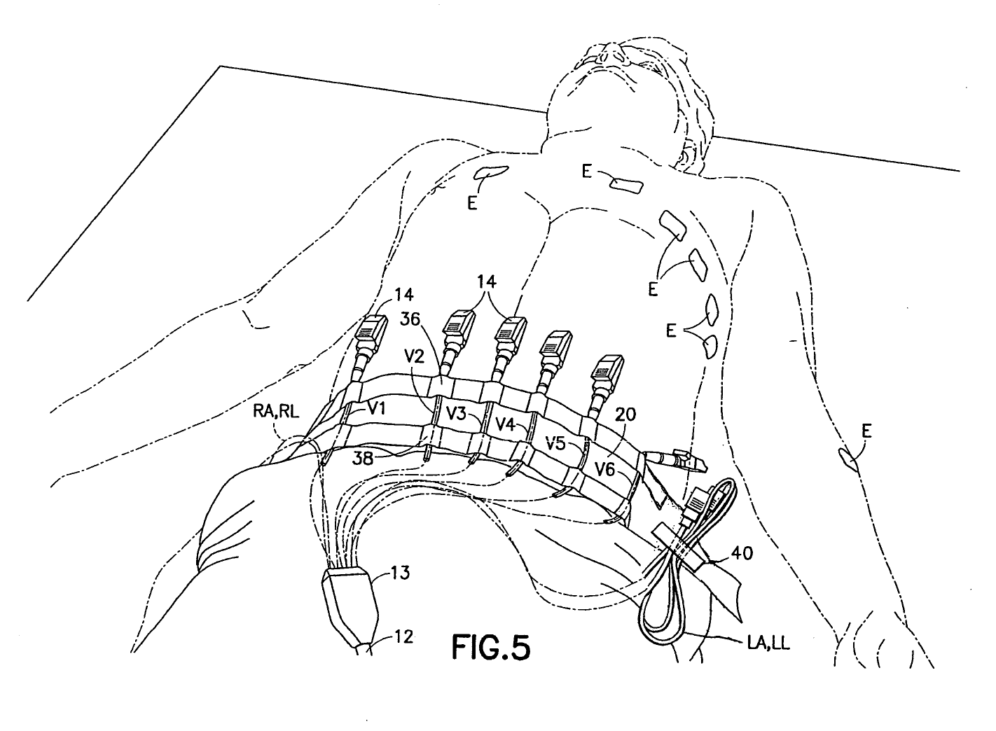

FIG. 6 is a perspective view showing the device of FIG. 5 after the leads have been attached to the patient and connected to the medical device.

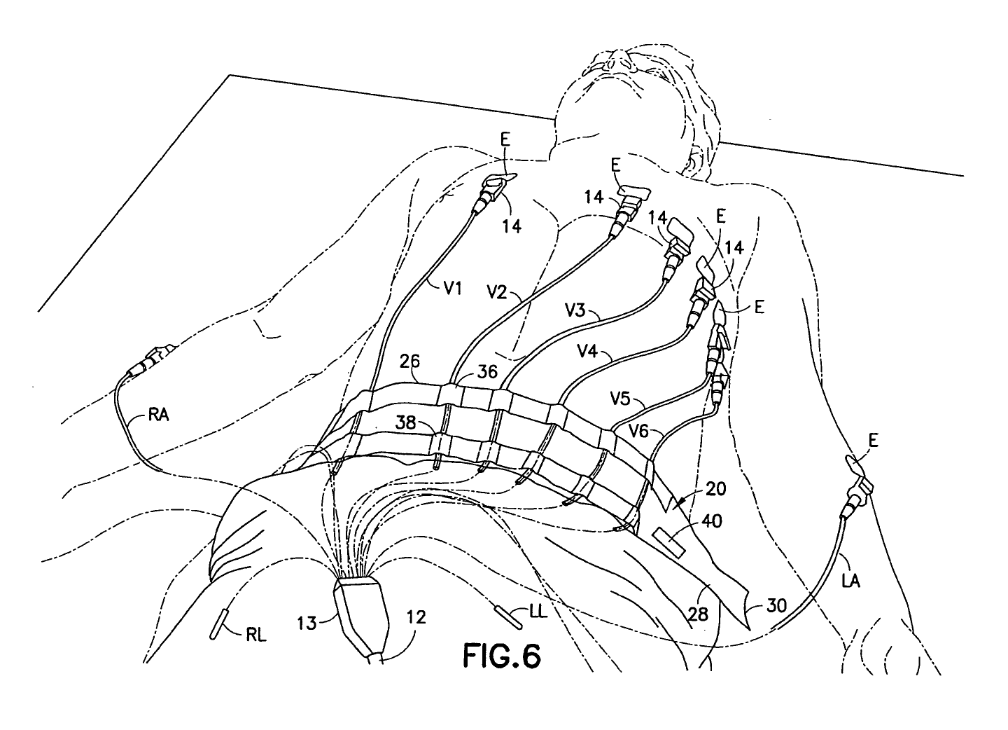

### DETAILED DESCRIPTION OF THE PREFERRED EMBODIMENT

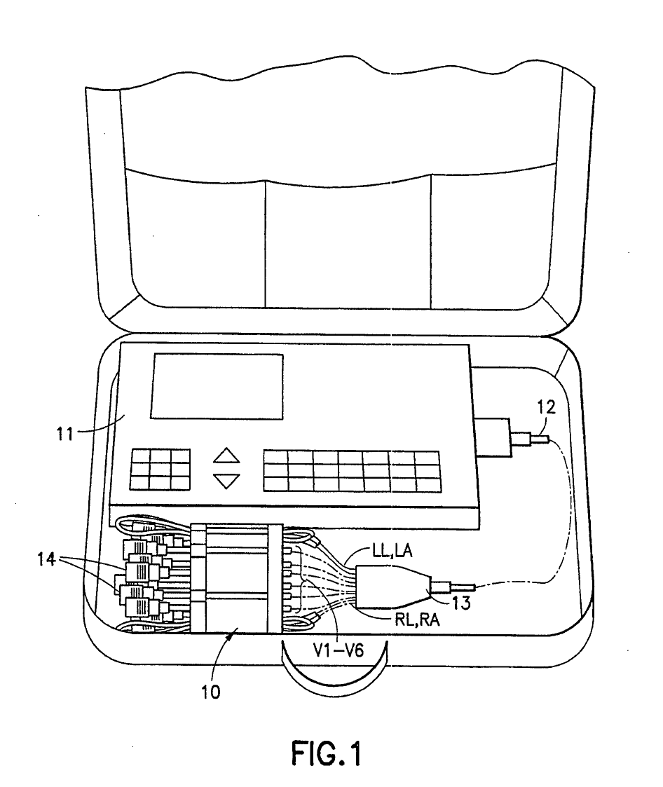

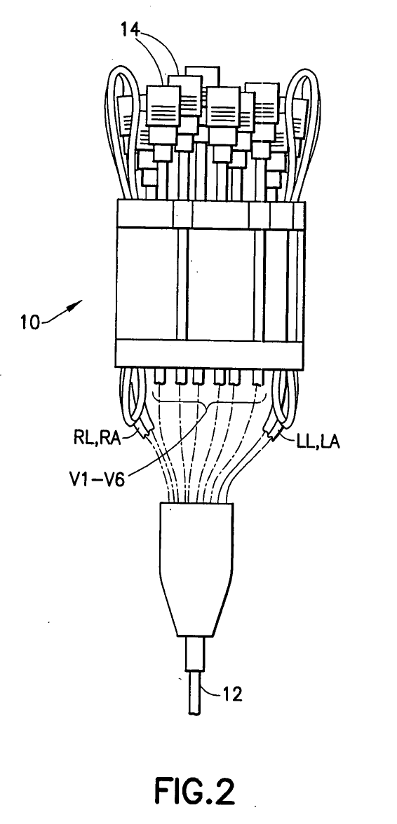

A lead alignment device in accordance with the subject invention is identified generally by the numeral 10 in FIGS. 1 and 2. The device 10 is intended for use with an EKG apparatus 11. The EKG apparatus 11 is shown merely in schematic form, and can take any form known in the prior art or developed in the future. A multi-conductor cable 12 extends from the EKG apparatus 11 to a junction box 13.

Ten leads extend from the junction box 13 and are identified by the alphanumeric codes V1-V6, LA, LL, RA and RL. The leads V1 and V2 are intended to be secured to the V1 and V2 electrodes disposed near the right and left breasts of the patient respectively. The leads V3-V6 extend sequentially to the left of the V2 electrode and are connected to the V3-V6 electrodes that extend around the left side of the patient. The LA lead is a ground lead connected to an electrode on the left arm of the patient. The LL lead also is a ground lead connected to an electrode on the left leg of the patient. Similarly, the RA lead is a ground lead connected to an electrode on the right arm of the patient and the RL lead is a ground lead connected to an electrode on the right leg of the patient.

Connectors 14 are secured to the ends of the respective leads remote from the EKG apparatus 11. More particularly, the connectors 14 can take any form chosen by the manufacturer of the EKG apparatus 11 for connection to the electrodes that will be mounted on a patient. For example, the connectors 14 can generally take the form of an alligator clip or a clothespin that can be clipped elastically to a conductive region of the respective electrode. Alternatively, the connectors 14 can be configured to snap into engagement with posts on the corresponding electrode.

The alignment device 10 includes a flexible soft support 20, preferably formed from a vinyl sheet or a synthetic fabric. The flexible support 20 has a lower surface 22 that can be draped on the patient or on a pad disposed on the patient. The support 20 also has an upper surface 24 that will face away from the patient. The support 20 is substantially rectangular and includes a long superior edge 26 and a long inferior edge 28 that extend substantially parallel to one another. The support 20 further includes a left edge 30 and a right edge 32 that extend parallel to one another at opposite left and right ends of the support 20. The superior and inferior edges 26 and 28 define lengths “L” sufficient to extend completely across the chest or abdomen of the patient, and typically the length “L” will be in the range of 20-36 inches. The left and right end edges 30 and 32 are much shorter than the superior and inferior edges 26 and 28 and preferably define a width ‘W’ of about 3-6 inches.

Superior and inferior slidable attachment tunnels 36 and 38 respectively are secured to the upper surface 24 of the support. More particularly, the superior slidable attachment tunnels 36 define a substantially linear array extending parallel to and substantially adjacent the superior edge 26 of the support 20. Similarly, the inferior slidable attachment tunnels 38 define a linear array extending substantially parallel to and substantially adjacent the inferior edge 28 of the support 20. As a result, the array of superior slidable attachment tunnels 36 and the array of inferior slidable attachment tunnels 38 extend substantially parallel to one another and are spaced from one another by a distance of about three inches. Furthermore, the respective superior slidable attachment tunnels 36 and inferior slidable attachment tunnels 38 are aligned in pairs so that the superior slidable attachment tunnel 36 and the inferior slidable attachment tunnel 38 in each pair extend along a line perpendicular to the superior and inferior edges 26 and 28 and hence parallel to the opposite left and right edges 30 and 32.

The superior and inferior slidable attachment tunnels 36 and 38 each are configured for releasably and slidably receiving one of the leads V1-V6 respectively. Thus, each of the leads can be slid substantially longitudinally to the axis of the respective lead through the corresponding pair of superior and inferior slidable attachment tunnels 36 and 38. The dimensions of the respective slidable attachment tunnels 36 and 38, however, do not permit the connectors 14 to pass through. Furthermore, the attachment tunnels 36 and 38 are dimensioned and configured to exert frictional resistance or drag on the leads V1-V6. Thus, the leads V1-V6 can be slid through the attachment tunnels 36, 38 in response to a pulling force by the technician. However, free unintended movement is resisted.

In a preferred embodiment, the superior and inferior slidable attachment tunnels 36 and 38 are formed from a flexible fabric-like material. For example, the slidable attachment tunnels 36 and 38 can be formed from mateable pairs of hook and loop strips, such as those sold under the trademark Velcro. Lower strips 36L, 38L of Velcro are permanently sewn or otherwise secured to the support 20 so that the loops of the Velcro strips face away from the support 20. In a preferred embodiment, the lower strips 36L, 38L are sewn to the support 20 along the longitudinal edges and at the opposite longitudinal ends of the lower strips 36L, 38L. However, in other embodiments, the lower strips 36L, 38L may be attached by adhesive, sonic welding or the like. Six superior upper strips 36U and six inferior upper strips 38U of a mating Velcro material then are secured to the lower strips 36L, 38L so that the hooked surface of the upper strips 36U, 38U face the looped surface of the respective lower strip 36L or 38L. Each upper strip 36U, 38U preferably defines a length of 2-3 inches to provide sufficient surface area on opposite sides of the lead for engaging the lower strip 36L, 38L with an adequate holding force. The attachment of the upper strips 36U, 38U to the lower strips 36L, 38L preferably relies entirely on the hooks and loops of the Velcro. However, in other embodiments, one end of each upper Velcro strip 36U, 38U may be sewn or otherwise permanently secured to the respective lower Velcro strip 36L, 38L. The slidable attachment tunnels 36, 38 can be adapted easily to leads V1-V6 of different cross-sections and/or to differently dimensioned connectors 14 merely by varying the extent of the attachment of the upper Velcro strip 36U, 38U to the respective lower Velcro strip 36L, 38L by pressing larger or smaller areas of the upper strip 36U, 38U onto the lower strip 36L, 38L. Thus, the leads V1-V6 are assured of being slidable in the respective tunnels 36, 38. Additionally, the loops and hooks of the Velcro exhibit some frictional resistance against the leads V1-V6 to prevent unintended sliding movement of the leads V1-V6 (e.g. in response to gravity). The slidable attachment tunnels 36, 38 will be opened only to replace a lead or to replace the entire lead alignment device 10. As a result, the slidable attachment tunnels 36 and 38 will be opened infrequently. Accordingly, the six superior upper strips 36U and six inferior upper strips 38U described above can be replaced by a single superior upper strip and a single inferior upper strip. With this embodiment, the V1-V6 leads are arranged sequentially on the lower strips 36L, 38L. The single superior upper strip 36U the single inferior upper strip 38U then are secured to the lower strips 36L, 38L to hold the leads V1-V6 in their designated positions, while permitting sliding movement of the leads V1-V6 along their longitudinal directions. This latter embodiment has lower manufacturing costs.

The device 10 further includes left and right ground lead holders 40 and 42 disposed respectively near the left and right side edges 30 and 32 of the support 20 and approximately midway between the superior and inferior edges 26 and 28. The ground lead holders 40 and 42 are flexible strips with non-metallic snaps or more preferably with Velcro attachments for releasably holding the ground leads LA, LL, RA and RL in a neat looped arrangement on the support 20. Accordingly, portions of each of the looped ground leads LA, LL, RA, RL that extend beyond the holder 40 or 42 will be supported on the support 20. In the preferred embodiment, each ground lead holder 40 and 42 has a lower Velcro strip sewn or otherwise permanently secured to the support 20. Each ground lead holder 40 and 42 further includes an upper Velcro strip releasably secured to the lower Velcro strip by the hooks and loops of Velcro material. Neither end of the upper Velcro strip of the ground lead holders 40 and 42 is sewn or otherwise permanently secured to the lower Velcro strip. Thus, the technician can open the respective ground lead holders 40 and 42 from either side in accordance with the particular characteristics of the patient, constraints of the test location or the preference of the technician. In an alternate embodiment, the ground lead holders may both be near the same side edges 30 or 32 of the support 20 to minimize reaching by the technician. Alternatively, a total of four ground lead holders can be provided (e.g., two at each side of the support 20) so that each ground lead LA, LL, RA and RL would have its own holder.

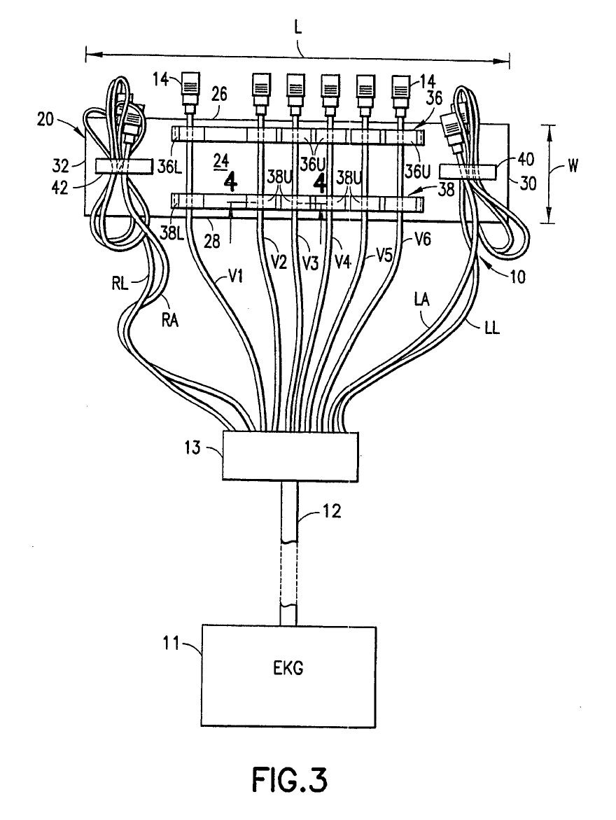

The leads V1-V6 can be engaged slidably in the respective slidable attachment tunnels 36 and 38 so that the leads V1-V6 are arranged sequentially, as shown in FIG. 3, to allow proper alignment for use with a patient as described herein. The leads V1-V6 then are slid in the slidable attachment tunnels 36, 38 so the connectors 14 are substantially adjacent the superior slidable attachment tunnels 36. The ground leads LA, LL, RA and RL then are looped and secured in the ground lead holders 40 and 42. The support 20 then can be folded or wrapped into a loose coil so that the leads are brought closer together. This assembly of the loosely wrapped support 20 and the orderly aligned leads then can be wrapped around itself or around the EKG apparatus 11. This orderly assembly of the lead alignment device 10, the leads and the EKG apparatus 11 can be placed by the medical technician in an appropriate carrying case or on a cart for storage and transport to a location at which a physical will be performed.

At an appropriate point during the physical, the technician will place the electrodes E at the proper positions on the chest, arms and legs of the patient. The technician then may place a clean pad, paper or the like across the abdomen of the patient. The pad ensures that the device 10 will not be placed directly on the exposed skin of any patient, and hence prevents bacteria or the like from being transferred from one patient to another. The technician then unwinds the device 10 and places the lower surface 22 of the flexible support 20 on the pad, paper or the like that has been placed on the abdomen of the patient so that the superior edge 26 of the flexible support 20 is closer to the patient's head. The technician then proceeds to grasp the connectors 14 one at a time, slides the leads V1-V6 longitudinally through the slidable attachment tunnels 36, 38 and sequentially connects the connectors 14 to the electrodes. The V1-V6 leads are arranged sequentially on the support 20 of the alignment device 10 and are at positions that will approximately align with the electrodes E. As a result, the technician can move quickly to join the respective connectors 14 to the electrodes E with a high degree of assurance that the leads V1-V6 are being connected properly. The LA and LL ground leads are released from the left ground lead holder 40 near the left side edge 30 of the support 20 and are connected to the electrodes E on the left arm and left leg of the patient. Similarly, the RA and RL ground leads are released from the right ground lead holder 42 near the right side edge 32 of the support 20 and are connected to electrodes E on the right arm and right leg of the patient.

The technician performs the EKG test in the conventional manner. Upon the completion of the test, the technician sequentially disconnects the six connectors 14 of the V1-V6 leads from the corresponding electrodes E on the chest of the patient. The V1-V6 leads then are pulled back through the slidable attachment tunnels 36 and 38 so that the connectors 14 substantially abut the superior slidable attachment tunnels 36. The LA and LL ground leads are disconnected from the electrodes E on the left arm and left leg of the patient and are looped for attachment in the left ground lead holder 40. Similarly, the RA and RL ground leads are disconnected from the electrodes on the right arm and right leg of the patient and are looped for attachment in the right ground lead holder 42. The flexible support 20 then can be rolled loosely onto itself and the assembled alignment device 10 and the leads can be stored independently of the EKG apparatus 11 or around the EKG apparatus 11. The alignment device 10 need not be separated from the respective leads, thereby allowing the leads to be retained in proper alignment for the next use of the EKG apparatus 11. The neatly stored EKG apparatus 11, the leads and the alignment device 10 then can be placed back in the carrying case or placed on a cart for transportation to the next physical.

The alignment device 10 described above and illustrated in the figures has several significant advantages. For example, once the alignment device 10 is assembled initially, the device is ready for repeated use. All leads are held in an organized, pre-assembled manner, with the chest leads V1-V6 in the correct sequential order to reduce the potential for erroneous lead placement during exam. This pre-assembly of the wire leads V1-V6, LA, LL, RA and RL on the flexible support 20 keeps the leads neat and eliminates or greatly reduces the potential for tangling of the leads and lead cross-over when setting up and performing an EKG exam on a patient. This significantly reduces the time of set up, avoids frustration for the technician and increases efficiencies.

The alignment device 10 can be used on any size patient. The sliding or gliding capability of the chest leads V1-V6 through the slidable attachment tunnels 36, 38 allows the leads V1-V6 to reach the electrodes 11 at their point of placement on the patient. The technician merely needs to pull each lead V1-V6 an appropriate distance to reach the corresponding electrode E. After release from their respective holders on the support, the longer ground lead wires retain their full length reach to distant corresponding electrodes on the arms and legs of the patient.

The alignment device 10 can be used with different model EKG machines, with varying numbers of wire leads and with varying diameters of lead wires V1-V6 and connectors 14 due to the ability to adjust the cross-sectional dimensions of the attachment tunnels 36, 38. In this regard, embodiments that rely upon Velcro type of attachments merely require upper strips 36U, 38U to be pressed sufficiently into engagement with the lower strips 36L, 38L on opposite respective sides of the leads V1-V6 to accommodate the given cross-sectional dimensions of the leads V1-V6 employed with a particular model of EKG machine.

The slidable attachment tunnels 36 and 38 normally do not need to be opened. However, the tunnels 36, 38 can be opened easily to change wire leads for repair or maintenance, to replace one alignment device 10 with a new alignment device 10 or to clean the alignment device 10.

The alignment device 10 is well suited for use with entirely non-metallic materials. The preferred hook and loop attachments strips (e.g. Velcro) have no metallic components. Non-metallic components, such as snaps or clips, can be used in certain embodiments.

The illustrated embodiments show the alignment device 10 for use on a patient in a supine position. However, the alignment device also can be used for an EKG exam performed on a patient sitting in a substantially erect position. In this situation, the lower surface 22 of the flexible support 20 can be disposed on the lap of the patient. Alternatively, the inferior edge 28 of the flexible support 20 can be supported on the lap of the patient, with the lower surface 22 facing and substantially adjacent to the abdomen of the patient. In certain instances, however, a non-interfering (non-metallic) carrier can be draped around the neck of the patient, over the shoulders and to the abdomen. Thus, the carrier defines a flexible U-shaped arrangement. The ends of the arms of this U-shaped carrier can be configured to releasably engage the lower surface 22 of the flexible support 20. This attachment can be achieved by hook and loop attachment strips similar to those described above for the attachment tunnels 36, 38.

The alignment device can be stored easily between the uses. In this regard, the flexible support 20 can be rolled, folded and/or collapsed upon itself without disrupting the preassembled organization of the leads. This rolled, folded or collapsed alignment device 10 can be placed in a carrying case with a portable EKG machine or can be stored at an appropriate easily accessible location on a cart used in a medical facility. The alignment device 10 is light weight and easily transportable. Accordingly, the alignment device can be used for all mobile EKG machines, including those used for hospitals, ambulance squads, in-home care patients and mobile insurance examinations.

The flexible support 20, the slidable attachment tunnels 36, 38 and the ground lead holders 40, 42 all preferably are formed from a washable synthetic material. Hence, the alignment device 10 can be maintained in a clean state.

The above-described components of the alignment device all are low cost and readily available. Additionally, these components are well suited to low cost high speed manufacturing and assembly techniques.

The alignment device 10 provides a comfortable experience for the patient who is receiving an EKG exam. In particular, the alignment device 10 permits a fast, easy and efficient set up by the technician. The patient will not observe the technician fumbling with wires. Furthermore, the patient will spend less time in a partly dressed state. All parts of the preferred alignment device 10 can provide a comfortable engagement with the patient due to the light weight flexible characteristics of the various components of the alignment device 10.

While the invention has been described with respect to a preferred embodiment, it is apparent that various changes can be made without departing from the scope of the invention. In this regard, the device has been illustrated for use with an EKG apparatus that has a total of ten leads. However, the device can be adapted for use with an EKG apparatus or similar testing apparatus that has fewer than ten leads or more than ten leads.

The alignment device of the illustrated embodiment includes Velcro-type attachments for releasably holding the leads on the support for slidable movement of the respective leads along their longitudinal direction. However, other slidable attachments can be used, such as non-metallic clips, non-metallic eyelets or the like.

The invention has been described as being used with a gauze, pad or the like to be positioned between the alignment device 10 and the patient. However, the gauze, pad or other such sanitary measure can be eliminated in certain instances or can take other forms.

The slidable attachment tunnels also can be provided for ground leads.

Separate holders can be provided for each ground lead.

Labels can be provided near each slidable attachment tunnel and/or each ground lead holder to identify the respective leads.

The device can be used to organize wire leads from medical devises other than EKG devices, such as apparatus used for sleep apnea studies.

What is claimed is:

1. A lead alignment device for use with a medical apparatus that has a plurality of chest leads, each of said chest leads having an electrical connector secured to an end of the lead remote from the apparatus, the lead alignment device comprising: a flexible support and a plurality of lead alignment means for slidably holding each of the chest leads so that the chest leads can be slid relative to the lead alignment device, while preventing separation of the chest leads from the lead alignment device, whereby the device substantially aligns, organizes the chest leads of the apparatus and prevents entanglement of the leads.

2. The lead alignment device of claim 1, wherein each of said lead alignment means is cross-sectionally dimensioned to hold the respective lead snugly and to prevent movement of the connector associated with each of said leads through the lead alignment means.

3. The lead alignment device of claim 2, wherein the device comprises a first substantially linear array of lead alignment means for respectively accommodating the chest leads and a second substantially linear array of lead alignment means for respectively accommodating the chest leads at a location spaced from the lead alignment means in the first array.

4. The lead alignment device of claim 3, wherein the first and second arrays of lead alignment means are substantially parallel to one another.

5. The lead alignment device of claim 4, wherein each lead alignment means in the second array substantially aligns with a corresponding lead alignment means in the first array in a direction extending substantially perpendicularly between the first and second arrays.

6. The lead alignment device of claim 1, wherein the support is formed from a flexible sheet material.

7. The lead alignment device of claim 6, wherein the flexible sheet material is a synthetic fabric.

8. The lead alignment device of claim 1, wherein each of said lead alignment means comprises a first fabric material having a plurality of loops formed thereon and a second fabric material having a plurality of hooks formed thereon for releasable engagement with the loops of the first fabric material.

9. The lead alignment device of claim 1, wherein the medical apparatus further comprises a plurality of ground leads, the lead alignment device further comprising at least one ground lead organizer for releasably holding the ground leads in a looped array on the support.

10. The lead alignment device of claim 9, wherein the at least one ground lead organizer comprises at least two ground lead organizers.

11. The lead alignment device of claim 10, wherein the support has opposed superior and inferior edges and opposed left and right edges extending between the superior and inferior edges, the at least two ground lead organizers comprising at least one ground lead organizer in proximity to the left edge of the support and at least one ground lead organizer in proximity to the right edge of the support.

12. A lead alignment device preassembled with a medical apparatus that has a plurality of leads, each of said leads having an electrical connector secured to an end of the lead remote from the apparatus, the lead alignment device comprising: a flexible support formed from a flexible synthetic sheet material having opposite superior and inferior edges and opposite left and right end edges extending between the superior and inferior edges, a plurality of superior lead alignment tunnels disposed in a substantially linear array substantially adjacent the superior edge of the flexible support and a plurality of inferior lead alignment tunnels disposed in a substantially linear array substantially adjacent the inferior edge of the flexible support, the lead alignment tunnels in the superior array being aligned respectively with the lead alignment tunnels in the inferior array to define pairs of aligned lead alignment tunnels, each of the pairs of lead alignment tunnels snugly holding one of the leads so that the leads can be slid relative to the corresponding pair of lead alignment tunnels in response to a pulling force exerted on the respective lead, while preventing separation of the leads from the lead alignment tunnels, whereby the device substantially aligns and organizes the leads of the apparatus and prevents entanglement of the leads.

13. The lead alignment device of claim 12, wherein each of said lead alignment means comprises a first fabric material having a plurality of loops formed thereon and a second fabric material having a plurality of hooks formed thereon for releasable engagement with the loops of the first fabric material.

14. The lead alignment device of claim 13, wherein the electronic apparatus is an EKG apparatus, the plurality of leads being a plurality of chest leads, the EKG apparatus further comprising a plurality of ground leads, the lead alignment device further comprising at least one ground lead organizer for releasably holding the ground leads in a looped array on the support.

15. The lead alignment device of claim 14, wherein the at least one ground lead organizer comprises at least two ground lead organizers.

16. A lead alignment device of claim 15, wherein the ground lead organizers comprise first and second ground lead organizer disposed respectively in proximity to the respective left and right edges of the flexible support.

17. A method for carrying out an EKG test, the method using an EKG apparatus having a plurality of chest leads and ground leads extending therefrom, each said lead having a connector at an end of the lead remote from the EKG apparatus, a flexible support extending substantially transverse to the leads and slidably engaging each of the respective chest leads, the method comprising:
placing electrodes at selected locations on a patient;
placing the flexible support on the abdomen of the patient;
sequentially pulling the connectors of the chest leads away from the flexible support and to the respective electrodes so that the corresponding chest leads slide relative to the flexible support;
sequentially connecting the connectors of the chest leads to selected ones of the electrodes;
performing the EKG test with the EKG apparatus;
disconnecting the chest leads connectors from the respective electrodes; and
pulling the chest leads relative to the flexible support so that the corresponding connectors are moved back to positions substantially adjacent the flexible support.

18. The method of claim 17, wherein the flexible support includes retainers for holding the ground leads releasably in coiled arrays, the method further comprising releasing the ground leads from their retainers, uncoiling the ground leads and connecting the connectors of the ground leads to electrodes on arms and legs of the patient before performing the EKG test, and then disconnecting the connectors of the ground leads from the respective electrodes on the arms and legs, coiling the ground leads and releasably holding the ground leads in the retainers after performing the EKG test.

19. The method of claim 17, further comprising collapsing the flexible support onto itself for storage between EKG tests.

20. The method of claim 19, further comprising opening the collapsed flexible support prior to placing the flexible support on the patient during a subsequent EKG test.
